(19) United States
(12) Patent Application Publication
(10) Pub. No.: US 2025/0087309 A1
(43) Pub. Date: Mar. 13, 2025

(54) APPARATUS AND METHOD FOR PREDICTING ULTRA-HIGH TEMPERATURE PROPERTY

(71) Applicant: Foundation of Soongsil University-Industry Cooperation, Seoul (KR)

(72) Inventors: Jiwoong Kim, Seoul (KR); Hyokyeong Kim, Seoul (KR); Haeun Lee, Seoul (KR); Myungjae Kim, Seoul (KR)

(73) Assignee: Foundation of Soongsil University-Industry Cooperation, Seoul (KR)

(21) Appl. No.: 18/670,972

(22) Filed: May 22, 2024

(30) Foreign Application Priority Data
Sep. 13, 2023 (KR) 10-2023-0121599

(51) Int. Cl. G16C 20/30

(52) U.S. Cl. CPC G16C 20/30

(57) ABSTRACT

The present invention is to predict physical properties of a new composition, especially changes in physical properties at ultra-high temperatures, and has the effect of selecting compositions with desired physical properties without going through direct experiments by predicting changes in physical properties of new compositions, especially at ultra-high temperatures, predicting changes in physical properties due to changes in composition at absolute zero, and predicting changes in physical properties at ultra-high temperatures using the QHA (Quasi-harmonic approaching) technique and the Debye approximation for the stable phase of the composition.

### CROSS-REFERENCE TO RELATED APPLICATION

This application claims priority to and the benefit of Korean Patent Application No. 10-2023-0121599, filed on Sep. 13, 2023, the disclosure of which is incorporated herein by reference in its entirety.

### TECHNICAL FIELD

The present invention relates to technology for predicting the properties of materials, and particularly to technology for predicting deterioration of physical properties at ultra-high temperatures.

### BACKGROUND OF THE INVENTION

Materials such as ultra-high temperature ceramics are used in extreme environments such as national defense or aviation/space fields, and therefore must be durable without deteriorating at ultra-high temperatures of 2,000° C. or higher.

Therefore, for the development of these materials, it is essential to identify changes in the material's properties by simulating extreme environments such as ultra-high temperatures.

For this purpose, the related arts used a method of designing a material with excellent physical properties at room temperature and measuring changes in the material's physical properties through ultra-high temperature simulation experiments. In other words, an empirical method was used to confirm the physical properties through experiments by adding a specific content of elements known to be resistant to ultra-high temperatures to currently used ultra-high temperature materials.

However, it is realistically impossible to measure the gradual change in physical properties of the test piece using ultra-high temperature simulation experiments, and there is a limitation in that the degree of improvement should be determined or the direction of supplementation determined depending on the state of the test piece after the experiment is completed.

Therefore, trials and errors by numerous experiments and supplements were repeated to confirm the characteristics of ultra-high temperature ablation resistance, which requires not only time but also a huge cost to simulate ultra-high temperature environments.

The inventors of the present invention have completed the present invention capable of simple and efficient theoretical ultra-high temperature properties prediction after a long period of research efforts to solve the problems of the related arts as described above.

The present invention is derived from research supported by Korea Institute for Advancement of Technology (KIAT) grant funded by the Korea government (MTIE) [Project Unique Number: 1415182096, Project Number: P0021347(202211841923), Research Project Name: Establishment and verification of integrated DB for development of PVD thin film for processing high hardness, difficult-to-cut materials, Research Period: Jan. 1, 2023 to Dec. 31, 2023]

### SUMMARY OF THE INVENTION

### Technical Problem

In order to solve the problems of the related arts as described above, an object of the present invention is to provide an apparatus and method capable of predicting changes in physical properties at an ultra-high temperature in a theoretical environment rather than an ultra-high temperature simulation experiment.

The technical problems to be achieved in the present invention are not limited to the technical problems mentioned above, and other technical problems not mentioned will be clearly understood by those of ordinary skill in the art from the following description.

### Technical Solution

In order to solve the above problems, the apparatus for predicting ultra-high temperature properties according to a preferred embodiment of the present invention may include a control unit having one or more processors and memories, the control unit comprises: a modeling unit that calculates a change in physical properties of a composition according to changes in elements and composition at a predefined temperature; a stable phase prediction unit that predicts the most stable phase for each temperature for a composition of elements and composition selected according to the change in physical properties; a physical property deterioration prediction unit that predicts a change in physical properties as temperature increases with respect to the predicted stable phase of the composition; and an excellent composition selection unit that screens the prediction results of the stability phase prediction unit and the physical property deterioration prediction unit according to predetermined criteria to select a necessary composition.

The stable phase prediction unit or the physical property deterioration prediction unit may predict a stable phase for each temperature or a change in physical properties for each temperature using the Quasi-Harmonic Approximation (QHA) method.

The physical property deterioration prediction unit may predict physical property deterioration by the Debye approximation.

The physical property deterioration prediction unit may predict physical property deterioration by the Debye approximation only if the composition is a ceramic composition.

The predefined temperature may be absolute zero (OK).

The method for predicting ultra-high temperature properties according to another preferred embodiment of the present invention may include calculating a change in physical properties of a composition according to changes in elements and composition at a predefined temperature; predicting the most stable phase for each temperature for a composition of elements and composition selected according to the change in physical properties; predicting a change in physical properties as temperature increases with respect to the predicted stable phase of the composition; and screening the prediction results of the predicting a stable phase and the predicting a change in physical properties according to predetermined criteria to select a necessary composition.

The predicting a stable phase and the predicting a change in physical properties may predict a stable phase for each temperature or a change in physical properties for each temperature using the Quasi-Harmonic Approximation (QHA) method.

The predicting a change in physical properties may predict physical property deterioration by the Debye approximation.

The predicting a change in physical properties may predict physical property deterioration by the Debye approximation only if the composition is a ceramic composition.

The predefined temperature may be absolute zero (OK).

### Advantageous Effects

According to the present invention, it is possible to efficiently screen a material having desired physical properties without repeating trial and error in order to predict physical properties of an ultra-high temperature environment.

In addition, by conducting ultra-high temperature experiments only on such screened materials, there is an advantage of saving time and cost for ultra-high temperature experiments.

The effects of the present invention are not limited to those mentioned above, and other effects not mentioned will be clearly understood by those of ordinary skill in the art from the following description.

### BRIEF DESCRIPTION OF THE DRAWINGS

FIG. 1 is a schematic structural diagram of an apparatus for predicting ultra-high temperature properties according to a preferred embodiment of the present invention.

FIG. 2 is a detailed structural diagram of a control unit of the apparatus for predicting ultra-high temperature properties according to a preferred embodiment of the present invention.

FIGS. 3 to 7 show examples of implementation results of the apparatus for predicting ultra-high temperature properties according to a preferred embodiment of the present invention.

FIG. 8 is a schematic flow chart of a method for predicting ultra-high temperature properties according to another preferred embodiment of the present invention.

### DETAILED DESCRIPTION OF THE EMBODIMENTS

The above-mentioned objects, means, and effects thereof of the present invention will become more apparent from the following detailed description in relation to the accompanying drawings, and accordingly, those skilled in the art to which the present invention belongs will be able to easily practice the technical idea of the present invention. In addition, in describing the present invention, when it is determined that a detailed description of a related known technology may unnecessarily obscure the subject matter of the present invention, the detailed description will be omitted.

The terms used in this specification are for the purpose of describing embodiments only and are not intended to limit the present invention. In this specification, the singular forms “a,”, “an,” and “the” also include plural forms in some cases unless otherwise specified in the context. In this specification, terms such as “include”, “comprise”, “provide” or “have” do not exclude the presence or addition of one or more other elements other than elements mentioned.

In this specification, terms such as “or” and “at least one” may represent one of the words listed together or a combination of two or more thereof. For example, “A or B” and “at least one of A and B” may include only one of A or B, or may also include both A and B.

In this specification, descriptions according to “for example”, etc. may not exactly match the information presented, such as the recited properties, variables, or values, and effects such as modifications, including tolerances, measurement errors, limits of measurement accuracy, and other commonly known factors should not limit the modes for carrying out the invention according to the various exemplary embodiments of the present invention.

In this specification, when an element is described as being “connected” or “linked” to another element, it will be understood that it may be directly connected or linked to the other element but intervening elements may also be present. On the other hand, when an element is referred to as being “directly connected” or “directly linked” to another element, it will be understood that there are no intervening elements present.

In this specification, when an element is described as being “on” or “adjacent to” another element, it will be understood that it may be directly “on” or “connected to” the other element, but intervening elements may also be present. On the other hand, when an element is described as being “directly on” or “directly adjacent to” another element, it will be understood that there are no intervening elements present. Other expressions describing the relationship between the elements, for example, “between” and “directly between”, and the like can be construed similarly.

In this specification, terms such as “first” and “second” may be used to describe various elements, but, the above elements should not be limited by the terms above. In addition, the above terms should not be construed as limiting the order of each element, and may be used for the purpose of distinguishing one element from another. For example, a “first element” may be named as a “second element” and similarly, a “second element” may also be named as a “first element.”

Unless otherwise defined, all terms used in this specification may be used with meanings commonly understood by those of ordinary skill in the art to which the present invention belongs. In addition, terms defined in a commonly used dictionary are not interpreted ideally or excessively unless explicitly and specifically defined.

Hereinafter, preferred embodiments according to the present invention will be described in detail with reference to the accompanying drawings.

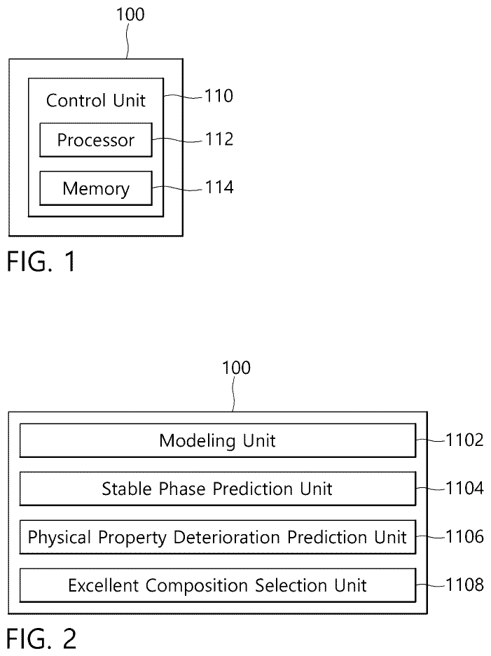

FIG. 1 is a schematic structural diagram of an apparatus for predicting ultra-high temperature properties according to a preferred embodiment of the present invention.

The apparatus 100 for predicting ultra-high temperature properties according to the present invention may include a control unit 110, and the control unit 110 may include one or more processors 112 and memories 114.

The control unit 110 controls the apparatus 100 for predicting ultra-high temperature properties and controls the performance of various functions for predicting physical properties. To this end, the control unit 110 may include, but is not limited to, a processor 112 that is hardware or a process that is software executed on the processor, or the like.

The memory 114 may store various information necessary for the operation of the apparatus 100 for predicting ultra-high temperature properties. The information stored in the memory 114 may include, but is not limited to, information for controlling the control unit 110, information for predicting physical properties by the control unit 110, information analyzed, program information related to a method for predicting ultra-high temperature physical properties, or the like.

For example, the memory 114 may include, but is not limited to, a hard disk type, a magnetic media type, a compact disc read only memory (CD-ROM), an optical media type, a magneto-optical media type, a multimedia card micro type, a flash memory type, a ROM type (read only memory type), or a RAM type (random access memory type) and the like depending on the type. In addition, the memory 114 may be a cache, a buffer, a main memory, an auxiliary memory, or a separately provided storage system depending on its purpose/location, but is not limited thereto.

FIG. 2 is a detailed structural diagram of a control unit of the apparatus for predicting ultra-high temperature properties according to a preferred embodiment of the present invention.

As described above, the control unit 110 may include one or more processors 112 and memories 114, and may include a modeling unit 1102, a stable phase prediction unit 1104, a physical property deterioration prediction unit 1106, and an excellent composition selection unit 1108.

The modeling unit 1102 predicts changes in various physical properties of the material through various changes in the additive element and composition.

For example, it changes the elements that are added at a predefined temperature, and predicts the physical properties and stability by predicting the tendency for the physical properties to vary by changing the composition ratio even if the added elements are the same.

In this case, the predefined temperature may be an absolute temperature of 0 degrees (OK) for accurate prediction of physical properties at room temperature rather than ultra-high temperature, but is not limited thereto.

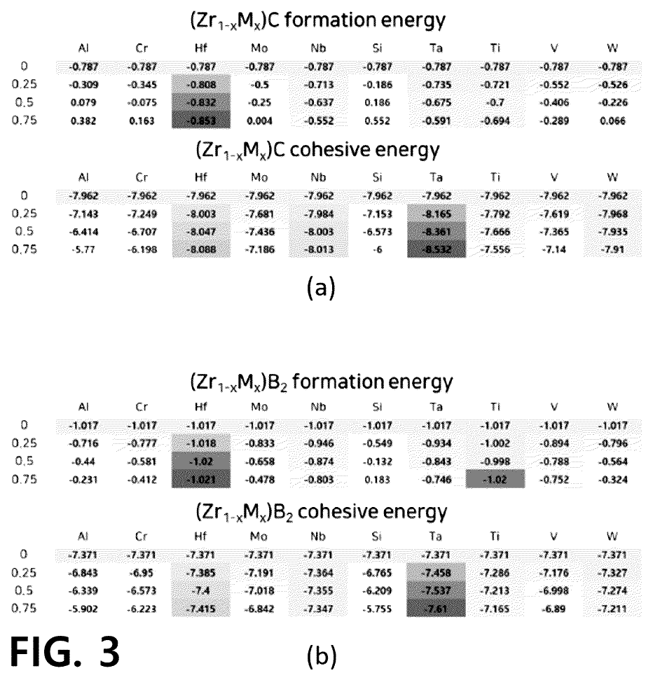

FIG. 3 is a diagram showing an example of physical properties that vary according to changes in an additive element and a composition by the modeling unit 1102.

The example in FIG. 3(a) shows a result of predicting changes in physical properties by changing the metal element M in the chemical formula (Zr₁₋ₓMₓ)C and adjusting X, which is the composition ratio of Zr and M.

The metal element M is replaced with various metal elements such as aluminum (Al), chromium (Cr), and titanium (Ti), and X is changed to 0, 0.25, 0.5, 0.75, etc. to change the composition.

In this way, the metal element M changes, and the formation energy, cohesive energy, etc. are calculated for the composition material whose composition changes depending on X.

Formation energy is a measure of the amount of energy entering and leaving during a chemical reaction, and cohesive energy is the energy required to separate one of the constituent atoms or molecules, so both can be used as a measure of the stability of the composition.

FIG. 3(b) is a result of calculating the formation energy and cohesive energy while changing M and X in the chemical formula (Zr₁₋ₓMₓ)B₂.

The stable phase prediction unit 1104 selects a stable phase by predicting the phase stability according to temperature for the composition whose stability is predicted according to changes in the added elements and composition in the modeling unit 1102.

Since the modeling unit 1102 predicts the physical properties and stability at room temperature or 0 K, the stable phase prediction unit 1104 predicts the phase stability according to temperature changes in order to predict the physical properties of these compositions at ultra-high temperatures.

To predict phase stability according to temperature, the Quasi-Harmonic Approximation (QHA) technique is used.

The QHA technique is a technique for calculating that phase stability varies depending on the volume even at the same temperature. Even though the exact change in physical properties cannot be predicted by the QHA technique, it has the advantage of being able to suggest the direction of the change in physical properties, and has the effect of being able to quickly select a group of candidates from a vast number of candidate compositions.

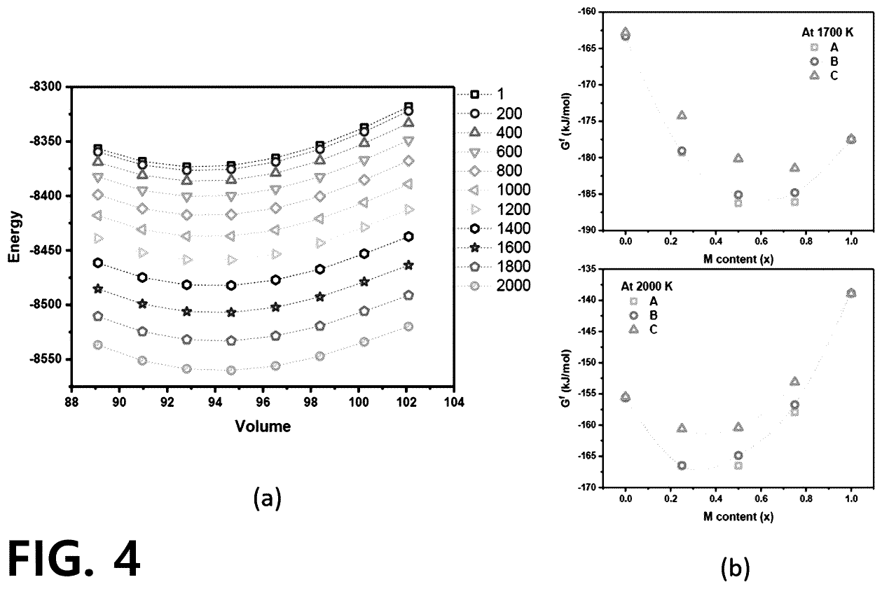

FIG. 4 is an example showing a result of such phase stability prediction.

FIG. 4(a) shows an example in which energy stability changes according to change in volume at various temperatures (1, 200, 400 . . . , 1800, 2000) even if the temperature does not change.

Therefore, using this QHA technique, the stable phase prediction unit 1104 predicts the stable phase at a predefined temperature according to changes in various added elements and composition ratios.

FIG. 4(b) shows a change in the formation Gibbs free energy according to a change (A, B, C) in composition elements and a change (M content (X)) in composition ratio at the same temperature (1700K or 2000K).

The physical property deterioration prediction unit 1106 predicts changes in the physical properties of the selected compositions at the desired temperature, that is, ultra-high temperature.

When an ultra-high temperature is applied to the composition, phase transition occurs or physical properties are deteriorated, and the physical property deterioration prediction unit 1106 predicts such high temperature deterioration using the QHA technique. Here, for faster calculations, the phonon lattice vibration contribution to the heat capacity can also be calculated using the Debye approximation, which replaces the phonon dispersion with a linear dispersion relation. When using the Debye approximation, more accurate results can be obtained from ceramic materials such as carbide/nitride.

Finally, the excellent composition selection unit 1108 databases the predicted results and derives the excellent composition that best suits the purpose. Although it is necessary to verify whether the derived composition actually has such properties through experiments, it is possible to quickly predict which materials are more stable and less deteriorated at ultra-high temperatures among compositions with numerous elements and composition ratios. Therefore, it has the advantage of significantly saving time and cost for experiments compared to the related arts.

Among the databased compositions, compositions with little or no phase transition and little deterioration of physical properties will be selected. However, if a phase transition necessarily occurs, selecting a composition with excellent physical properties in the state in which the phase transition occurs is more likely to result in a material that meets the purpose.

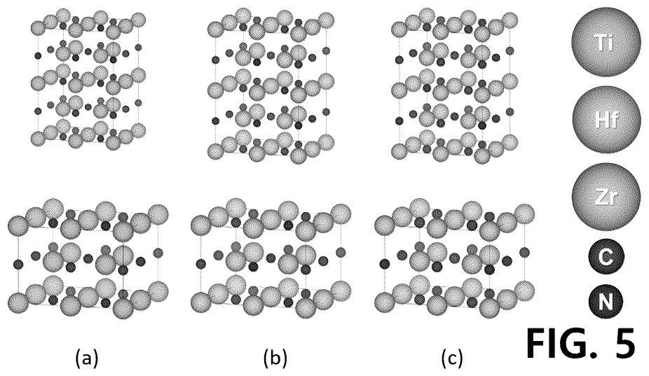

FIGS. 5 to 7 show an example in which ultra-high temperature physical properties are predicted by the apparatus for predicting ultra-high temperature properties according to the present invention.

FIG. 5 shows an example of cubic structure compositions modeled by the modeling unit 1102.

FIGS. 5(a), (b), and (c) show modeling of Ti(C_{0.5}N_{0.5}), Hf(C_{0.5}N_{0.5}), and Zr(C_{0.5}N_{0.5}), respectively.

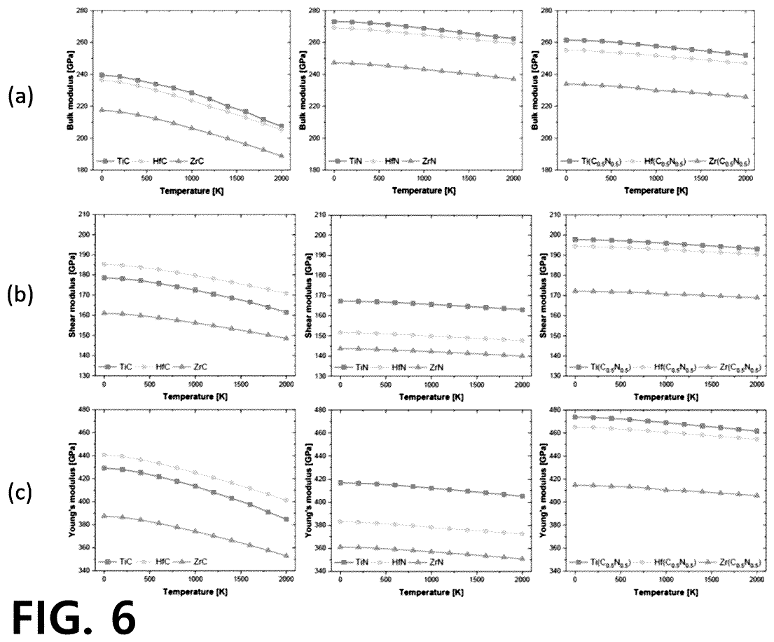

FIG. 6 shows an example of predicting the degree of deterioration of physical properties as temperature increases by the physical property deterioration prediction unit 1106.

FIG. 6(a) shows a result of predicting the degree of deterioration of bulk modulus as temperature increases.

FIG. 6(b) shows a result of predicting the degree of deterioration of the shear modulus as temperature increases and FIG. 6(c) shows a result of predicting the degree of deterioration of Young's modulus as temperature increases.

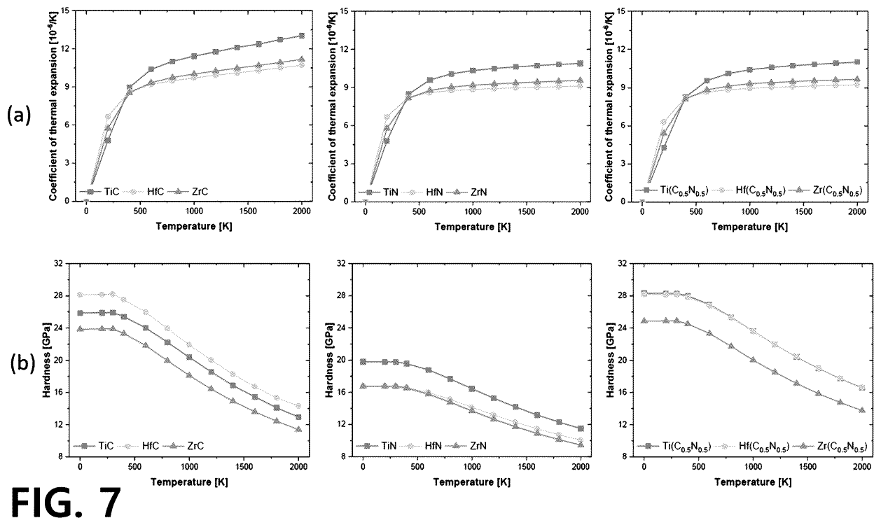

Finally, FIG. 7 shows the prediction of the coefficient of thermal expansion and the degree of deterioration of physical properties as temperature increases.

FIG. 7(a) shows a change in coefficient of thermal expansion as temperature increases.

FIG. 7(b) shows a change in hardness as temperature increases.

In this way, it is possible to quickly and efficiently screen compositions that are close to the desired physical properties for a large number of compositions by prediction through simulation rather than through experiment.

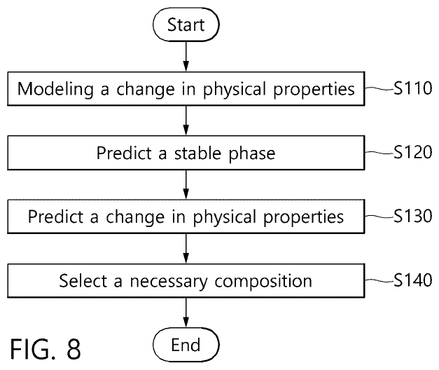

FIG. 8 is a schematic flow chart of a method for predicting ultra-high temperature properties according to another preferred embodiment of the present invention.

The method for predicting ultra-high temperature properties according to the present invention may be performed by a control unit including one or more processors and memories.

First, it models changes in various physical properties of the composition by changing the elements added or changing the composition at a predetermined temperature (S110).

The predetermined temperature may be OK, but is not limited thereto. First, it predicts the physical properties at OK or room temperature, and then predicts changes in the physical properties at ultra-high temperatures.

Next, it selects a stable phase by modeling changes in physical property changes according to element change and composition change and then predicting phase stability according to temperature (S120).

It predicts the physical properties and stability at OK and then predicts the phase stability at various temperatures to predict the physical properties at ultra-high temperatures. For this purpose, the QHA technique can be used.

After predicting the stable phase and selecting a composition that has a stable phase at a specific temperature, it predicts how the composition's physical properties change as temperature changes, that is, when it reaches an ultra-high temperature state (S130).

When an ultra-high temperature is applied to the composition, phase transition occurs or physical properties are deteriorated, and it predicts such high temperature deterioration using the QHA technique. As described earlier, here, for faster calculations, the phonon lattice vibration contribution to the heat capacity can also be calculated using the Debye approximation, which replaces the phonon dispersion with a linear dispersion relation.

Finally, it uses the results of predicting changes in physical properties at ultra-high temperatures to select an excellent composition suitable for the purpose (S140).

Even if these simulation results are not very accurate, they can suggest directions for composition selection and can significantly reduce actual experiments compared to the conventional method of obtaining the desired composition by repeating trial and error, which can save a lot of time and cost.

In the detailed description of the present invention, although specific embodiments have been described, it is apparent that various modifications are possible without departing from the scope of the present invention. Therefore, the scope of the present invention is not limited to the described embodiments, and should be defined by the following claims and their equivalents.

What is claimed is:

1. An apparatus for predicting ultra-high temperature properties, comprising:
a control unit having one or more processors and memories, the control unit comprises:
a modeling unit that calculates a change in physical properties of a composition according to changes in elements and composition at a predefined temperature;
a stable phase prediction unit that predicts the most stable phase for each temperature for a composition of elements and composition selected according to the change in physical properties;
a physical property deterioration prediction unit that predicts a change in physical properties as temperature increases with respect to the predicted stable phase of the composition; and
an excellent composition selection unit that screens the prediction results of the stability phase prediction unit and the physical property deterioration prediction unit according to predetermined criteria to select a necessary composition.

2. The apparatus for predicting ultra-high temperature properties of claim 1, wherein the stable phase prediction unit or the physical property deterioration prediction unit predicts a stable phase for each temperature or a change in physical properties for each temperature using the Quasi-Harmonic Approximation (QHA) method.

3. The apparatus for predicting ultra-high temperature properties of claim 2, wherein the physical property deterioration prediction unit predicts physical property deterioration by the Debye approximation.

4. The apparatus for predicting ultra-high temperature properties of claim 3, wherein the physical property deterioration prediction unit predicts physical property deterioration by the Debye approximation only if the composition is a ceramic composition.

5. The apparatus for predicting ultra-high temperature properties of claim 1, wherein the predefined temperature is absolute zero (OK).

6. A method for predicting ultra-high temperature properties, performed by a control unit comprising one or more processors and memories, the method comprising:
calculating a change in physical properties of a composition according to changes in elements and composition at a predefined temperature;
predicting the most stable phase for each temperature for a composition of elements and composition selected according to the change in physical properties;
predicting a change in physical properties as temperature increases with respect to the predicted stable phase of the composition; and
screening the prediction results of the predicting a stable phase and the predicting a change in physical properties according to predetermined criteria to select a necessary composition.

7. The method for predicting ultra-high temperature properties of claim 6, wherein the predicting a stable phase and the predicting a change in physical properties predicts a stable phase for each temperature or a change in physical properties for each temperature using the Quasi-Harmonic Approximation (QHA) method.

8. The method for predicting ultra-high temperature properties of claim 7, wherein the predicting a change in physical properties predicts physical property deterioration by the Debye approximation.

9. The method for predicting ultra-high temperature properties of claim 8, wherein the predicting a change in physical properties predicts physical property deterioration by the Debye approximation only if the composition is a ceramic composition.

10. The method for predicting ultra-high temperature properties of claim 6, wherein the predefined temperature is absolute zero (OK).
